(19) United States
(12) Patent Application Publication
(10) Pub. No.: US 2008/0269770 A1
(43) Pub. Date: Oct. 30, 2008

(54) Intraocular Lens Injector Subassembly

(76) Inventors: Joel Pynson, Toulouse (FR); Brian D. Rathert, Largo, FL (US); David P. Pollock, Victor, NY (US)

Correspondence Address: Bausch & Lomb Incorporated, One Bausch & Lomb Place, Rochester, NY, 14604-2701, US

(21) Appl. No.: 12/132,969

(22) Filed: Jun. 4, 2008

Related U.S. Application Data
(63) Continuation-in-part of application No. 11/615,430, filed on Dec. 22, 2006.

(51) Int. Cl. A61F 9/007

(52) U.S. Cl. 606/107

(57) ABSTRACT

An IOL injector subassembly, comprising a lens cartridge, an IOL, and a fastener connected to the lens cartridge arranged to maintain the IOL in a position disposed over at least a portion of the first lumen segment and over at least the second lumen segment. A method for facilitating loading of an IOL injector, comprising removing the fastener, and rotating the first portion relative to the second portion to fold the IOL.

### CROSS-REFERENCE

This application is a continuation-in-part of application Ser. No. 11/615,430, filed Dec. 22, 2006.

### FIELD OF THE INVENTION

The present invention relates to intraocular lens injector subassemblies, and more particularly to preloaded injector subassemblies.

### BACKGROUND OF THE INVENTION

IOLs are artificial lenses used to replace natural crystalline lenses of patients' when their natural lenses are diseased or otherwise impaired. Under some circumstances a natural lens may remain in a patient's eye together with an implanted IOL. IOLs may be placed in either the posterior chamber or the anterior chamber of an eye.

IOLs come in a variety of configurations and materials. Various instruments and methods for implanting such IOLs in an eye are known. Typically, an incision is made in a patient's cornea and an IOL is inserted into the eye through the incision. In one technique, a surgeon uses surgical forceps to grasp the IOL and insert it through the incision into the eye. While this technique is still practiced today, more and more surgeons are using IOL injectors, which offer advantages such as affording a surgeon more control when inserting an IOL into an eye and permitting insertion of IOLs through smaller incisions. Relatively small incision sizes (e.g., less than about 3 mm) are preferred over relatively large incisions (e.g., about 3.2 to 5+ mm) since smaller incisions have been attributed with reduced post-surgical healing time and reduced complications such as induced astigmatism.

In order for an IOL to fit through a small incision, it is typically folded and/or compressed prior to entering the eye where it will assume its original unfolded/uncompressed shape. Since IOLs are very small and delicate articles of manufacture, great care is taken in their handling, both as they are loaded into an injector and as the lenses are injected into patients' eyes.

It is desirable that an IOL be expelled from the tip of the IOL injector and into the eye in an undamaged condition and in a predictable orientation. Should an IOL be damaged or expelled from the injector in an incorrect orientation, a surgeon may need to remove or further manipulate the IOL in the eye, possibly resulting in trauma to the surrounding tissues of the eye. To achieve proper delivery of an IOL, consistent loading of the IOL into the injector device with a limited opportunity for damaging the IOL is desirable.

Various IOL injectors and other devices have been proposed and produced which attempt to address issues related to loading hydrophilic and/or hydrophobic IOLs into injectors, yet there remains a need for IOL injector components and loading techniques that facilitate injector loading in a surgery room, packaging at manufacture, and robustness during shipping.

### SUMMARY

Aspects of the present invention are directed to an IOL injector subassembly, comprising (A) a lens cartridge comprising (i.) a first portion comprising a first lumen segment, and (ii.) a second portion comprising a second lumen segment, the second portion being rotatably connected to the first portion, (B) an IOL, and (C) a fastener connected to the lens cartridge arranged to maintain the IOL over the first lumen segment and over the second lumen segment.

In some embodiments, the first portion and the second portion are configured to be capable of forming a first lumen, the sub-assembly further comprising a proximal portion fixedly coupled to the second portion and having a second lumen therethrough, the first lumen being aligned with a distal end of the second lumen. The proximal end of the second lumen may be funnel-shaped.

In some embodiments, the fastener is connected to the cartridge at two locations.

In some embodiments, the fastener is connected to the cartridge at four locations. The cartridge may comprise an injector tip configured for entry into an incision in an eye.

The subassembly may be in a combination with a container, wherein the sub-assembly is disposed in the container. The container may include a cover to seal the subassembly in the container.

Another aspect of the invention is directed to an IOL injector cartridge, comprising a first portion comprising a first lumen segment, and a second portion comprising a second lumen segment. The second portion is rotatably connected to the first portion, and the first portion and the second portion form a first lumen therebetween upon rotation of the first portion relative to the second portion (e.g., rotation about a hinge connecting the first portion and the second portion). The proximal portion is fixedly coupled to the second portion and having a second lumen therethrough, the first lumen being aligned with the second lumen. In some embodiments, the proximal end of the second lumen is funnel-shaped. The cartridge may comprise an injector tip configured for entry into an incision in the eye.

Another aspect of the invention is directed to a method for facilitating loading of an IOL injector, comprising providing an IOL injector subassembly, the subassembly comprising (I) a lens cartridge comprising (i.) a first portion comprising a first lumen segment, and (ii.) a second portion comprising a second lumen segment, the second portion being rotatably connected to the first portion. The subassembly also comprises an IOL, and a fastener connected to the lens cartridge arranged to maintain the IOL in a position over the first lumen segment and over the second lumen segment. The method also comprises removing the fastener; and rotating the first portion relative to the second portion to fold the IOL.

The method may further comprise loading the cartridge into a distal body portion of the injector. The method may further comprise attaching the distal body portion to a proximal body portion of the injector. Accordingly to some methods, the step of loading the cartridge into a distal body portion of the injector comprises loading the cartridge into the distal body portion using a handle removably attached to the cartridge.

The term “preloaded” as used herein means packaged together with and contacting an IOL.

The term “distal” as used herein refers to a feature closer to the location where an IOL is to be delivered to an eye.

The term “proximal” as used herein refers to a feature further from the location where an IOL is to be delivered to an eye.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1A is a perspective view of an example of an embodiment of an IOL injector subassembly including an IOL being maintained on a cartridge by a fastener;

FIG. 1B is a perspective view of the embodiment of a fastener shown in FIG. 1;

FIG. 2 is a perspective view of the IOL disposed on the cartridge of FIG. 1 after removal of the fastener;

FIG. 3 is a perspective view of the cartridge of FIG. 1 after the cartridge has been closed, the cartridge having the IOL (not shown) disposed inside in a folded state;

FIG. 4A is a perspective view of an example of an embodiment of an injector for use with a cartridge as shown in FIG. 1, the injector is in a disassembled state;

FIG. 4B is an expanded view of an example of a plunger tip suitable for use with the injector of FIG. 4A;

FIG. 5 is a perspective view of the injector of FIG. 4 in an assembled state, ready to inject an IOL into an eye;

FIG. 6 is a perspective view of a partially assembled injector illustrating the cartridge in a closed state and inserted into the proximal end of the distal portion of the injector of FIG. 4;

FIG. 7 is a front view of a container having the subassembly in FIG. 1 disposed inside; and

FIG. 8 is a side view of the container of FIG. 7.

### DETAILED DESCRIPTION

Aspects of the present invention are directed towards an IOL injector subassembly comprising a lens cartridge and a fastener connected to the cartridge arranged to maintain an IOL on the cartridge. In some embodiments, the subassembly is configured to facilitate loading of an IOL injector with an IOL by limiting the manipulation of the subassembly by surgical staff to: (1) removal of the fastener, (2) closing of the cartridge to fold the IOL, and (3) connection of the cartridge with remaining components of an injector. According to some embodiments, all of the steps may be accomplished without surgical staff directly contacting the IOL.

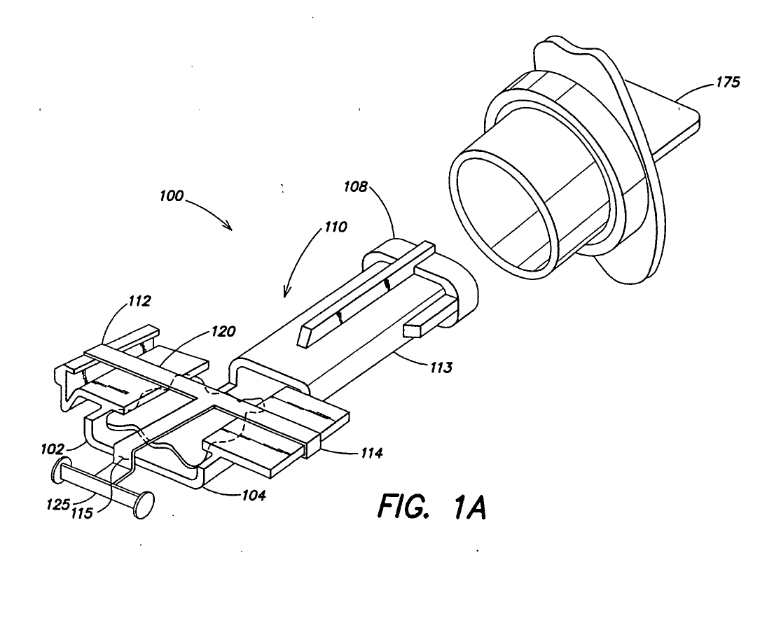

FIG. 1A is a perspective view of an example of an embodiment of an IOL injector subassembly 100 according to aspects of the invention. The subassembly comprises a lens cartridge 110 and a fastener 120 connected to the lens cartridge configured and arranged to maintain (e.g., hold) an IOL 150 on the cartridge, and to maintain the cartridge in an open state (i.e., a non-closed).

FIG. 1B is a perspective view illustrating fastener 120 in greater detail. The illustrated example of a fastener includes a cross member 122, having connectors 123a and 123b configured to connect to the cartridge. The fastener also includes a vertical member 124 connected to the cross member. The vertical member may be connected to a pull tab 125 that facilitates removal of the fastener when the IOL is to be folded. Pull tap 125 includes end caps 151a and 151b to guide a user's fingers to the pull tab.

As illustrated in FIG. 1A, the fastener is configured to be removably connected to the cartridge at a first location 112 and at a second location 114 using cross member 122. The cross member is rigid to overcome forces encountered by the subassembly that would tend to close the cartridge. It will be apparent that, in the illustrated embodiment, fastener 120 maintains the IOL on the cartridge by constraining the IOL from movement in a direction away from the cartridge. As illustrated, in some embodiments, a fastener comprises a vertical member 124 that includes a step feature 115 arranged to constrain the IOL from downward movement. As shown in FIG. 8, in some embodiments, the step feature permits the fastener to wrap around the bottom of the cartridge. It will be appreciated that a fastener need not have a vertical member. Referring again to FIG. 1A, the cartridge may be configured such that a proximal portion 113 constrains the IOL from movement upward. A fastener may have any suitable shape that maintains the IOL on the cartridge and maintains the cartridge in a non-closed condition. Preferably, the IOL is maintained in a non-stressed (i.e., non-folded) configuration when being maintained on the cartridge. In some embodiments, the fastener may consist of a single cross member, extending across (e.g., perpendicular to) the hinge; alternatively the fastener may consist of a single vertical member provided that the vertical member straddles the hinge.

Although the embodiment of the fastener illustrated in FIGS. 1-3 includes a vertical member 124 and a cross member 122, subassemblies including other fasteners that are configured to attach to the cartridge and maintain the IOL in place are within the scope of aspects of the invention. For example, the fastener may have a second cross member (not shown), parallel to cross member 122 also configured to contact and maintain the IOL on the cartridge. Alternatively, rather than having a vertical member and a cross member in a “T” shape when attached to the cartridge, the fastener may have an “X-shape” when attached to the cartridge and may be attached at to the cartridge at four locations.

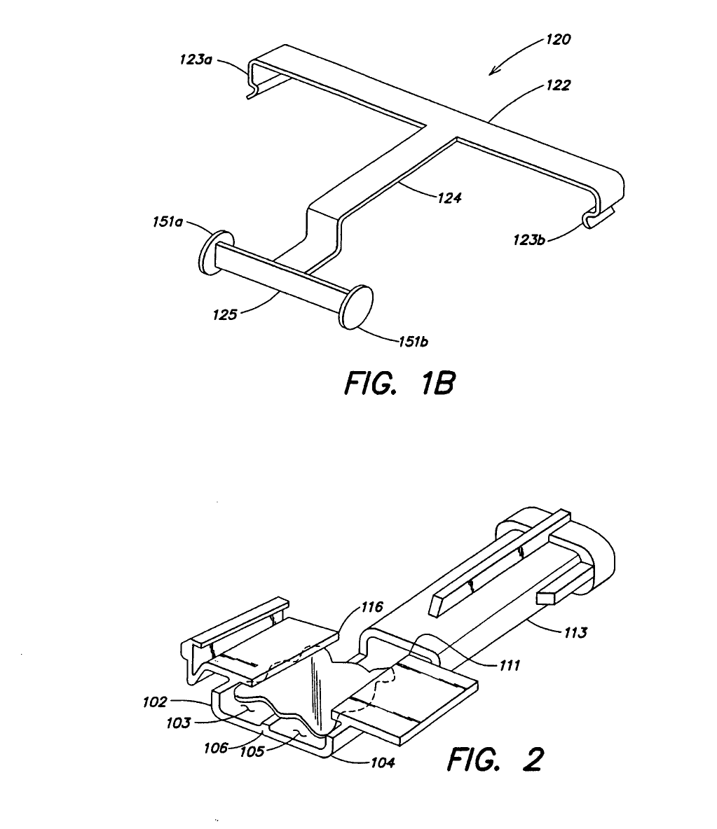

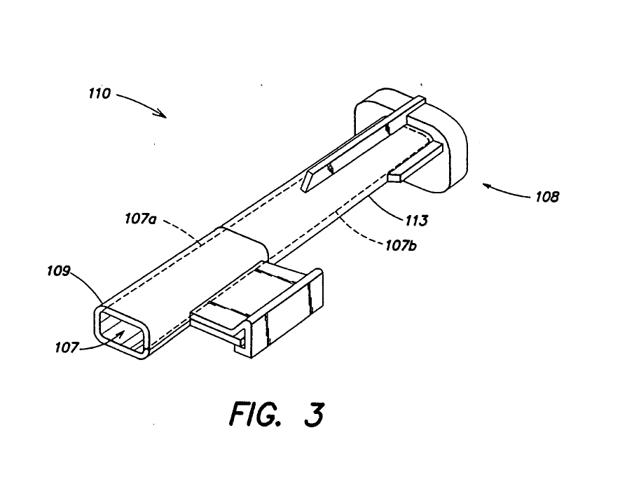

FIG. 2 is a perspective view of the IOL disposed on cartridge 110 after removal of the fastener 120. The cartridge may be any suitable conventional cartridge. However, FIGS. 1 and 2 illustrate one embodiment of a cartridge according to aspects of the present invention. The lens cartridge includes a first portion 102 comprising a first lumen segment 103, and a second portion 104 comprising a second lumen segment 105. The second portion is connected to the first portion by a hinge 106. A lumen portion 107a (shown in FIG. 3) is formed between the first lumen portion and the second lumen portion upon rotation about the hinge. Proximal portion 113 includes a cartridge lumen portion 107b therethrough (shown in FIG. 3). The lumen portion 107b through portion 113 is aligned with lumen portion 107a such that a plunger can extend therethrough. The hinge may be constructed such that the angle between the first portion and the second portion can reach an angle of no more than 180 degrees when in an open state. As described in greater detail below with reference to FIG. 6, proximal portion 113 facilitates assembly and/or operation of an injector by providing a guide to a plunger tip during assembly and/or operation. Proximal portion 113 is fixed to second portion 104.

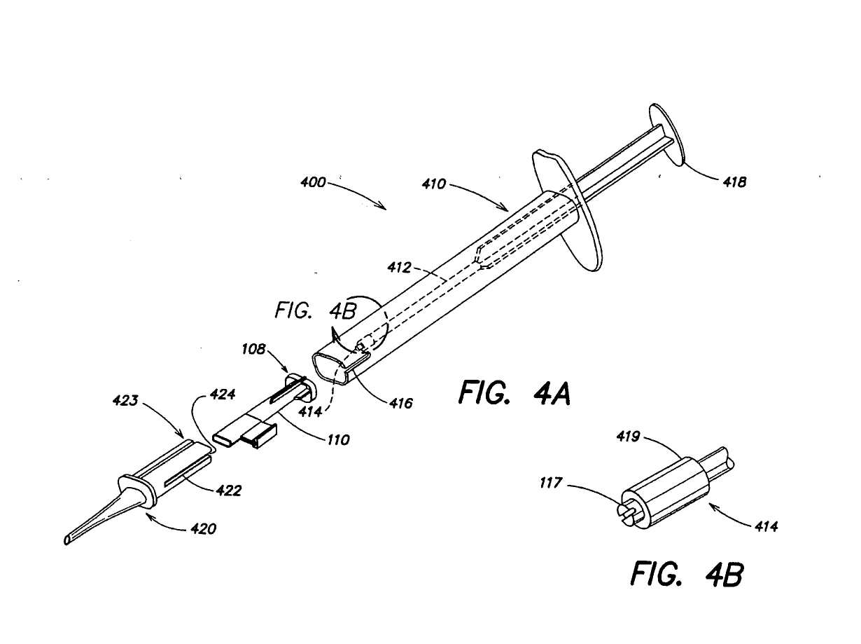

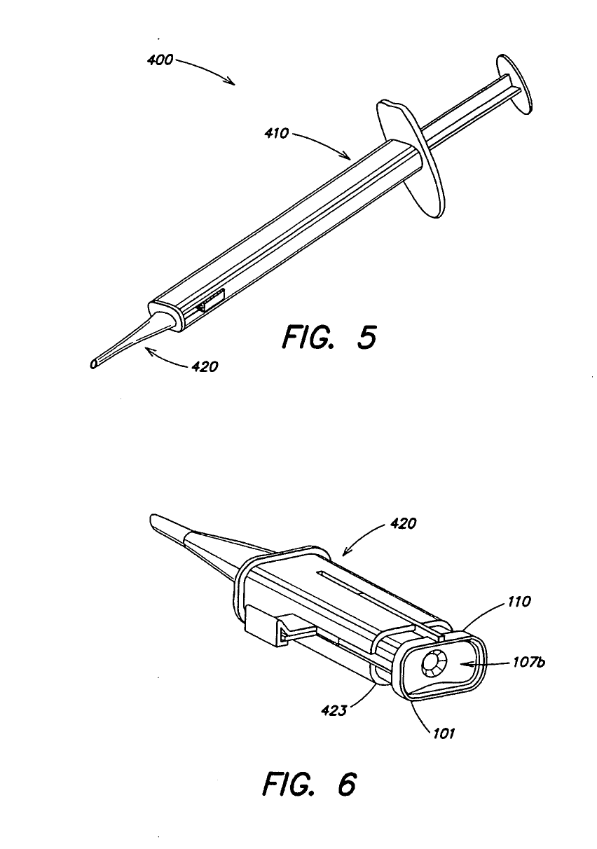

It will be appreciated that the IOL is maintained by fastener 120 in a position disposed over at least a portion of first lumen segment 103 and over at least a portion of the second lumen segment 105. The IOL straddles the hinge. After removal of the fastener, the hinge is freely actuatable, and upon rotating the first portion and second portion relative to one another, the IOL is manipulated into a folded state and the first lumen segment and the second lumen segment combine to form cartridge lumen portion 107a (shown in FIG. 3), as described above. Accordingly, after rotation, the IOL is folded and ready for insertion into a patient's eye upon connection of the cartridge with the remaining components of an injector as discussed below with reference to FIGS. 4A and 5. Typically, first portion 102 will be rotated, and second portion 104 (having proximal portion 113 fixed thereto) is held stationary for example using a holder 175 (shown in FIG. 1). However, it is relative motion of the first portion and the second portion that causes folding of the IOL, so the second portion 104 (having proximal portion 113 fixed thereto) can be rotated and first portion 102 held stationary.

FIG. 3 is a perspective view of cartridge 110 in a closed state, the IOL (not visible) being in a folded state within a lumen 107 of cartridge 110. Lumen 107 extends from distal end 109 to proximal end 108. As is described in greater detail below with reference to FIGS. 4A and 5, the cartridge is assembled into an injector for insertion of the IOL into an eye. The connection of the cartridge with the components occurs in a manner such that cartridge lumen 107 is aligned with injector lumen 424 (shown in FIG. 4B) and such that actuation of the injector (e.g., by pushing a plunger) delivers the IOL into a patient's eye.

As stated above, any suitable conventional cartridge may be used. For example, a conventional cartridge may include an injector tip for entry into an incision in an eye, and depositing of an IOL (but no proximal portion 113). As is most clearly visible in FIG. 2, in the illustrated embodiment of a cartridge, first portion 102 and second portion 104 may be provided with rails 116 and 111, respectively, that constrain the edges of IOL 150 prior to and during folding. Although not shown, in some embodiments of cartridges according to aspects of the present invention, a tip for entry into a relatively small incision in an eye may be provided along with proximal portion 113. The tip may be, for example, connected to the second portion of the cartridge. It will be appreciated that proximal portion 113 is relatively wide and is not suitable for entry into a relatively small incision in the eye.

Although in the illustrated embodiment, various features have been discussed that maintain the IOL on the cartridge when the fastener is connected to the cartridge, it will be appreciated that pressure on the lens caused by pressing the anterior surface and the posterior surfaces of the IOL between the fastener and the cartridge may be sufficient to maintain the lens.

FIG. 4A is a perspective view of an example of an embodiment of an injector in a disassembled state. The injector includes the cartridge 110 holding an IOL 150 (shown in FIG. 2), the cartridge being in a closed state. The cartridge is pushed into one of a proximal body portion 410 and a distal body portion 420 of the injector. When pushing cartridge 110 into the proximal body portion 410, care should be taken to allow the tip 414 of plunger 412 to enter the lumen 107 at the proximal end 108 of cartridge without damage.

Slots 416 and 422 are provided on the sides of the proximal body portion and the distal body portion, respectively, to permit first portion 102 and second portion 104 of the cartridge to be accommodated such that the proximal body portion receives the distal body portion. The proximal body portion and the distal body portion can thereby be connected together. Although the above example of an injector for use with subassemblies according to aspects of the present invention is a multipiece injector, other suitable injectors include side loaded (also referred to as breech loaded) injectors.

FIG. 5 is a perspective view of injector 400 of FIG. 4 in an assembled state ready to inject the IOL 150 (shown in FIG. 2) into a patient's eye. It will be appreciated that assembly of the injector is relatively simple, and direct manipulation of the IOL by surgical staff is not necessary.

As illustrated in FIG. 4B, in some embodiments, the plunger tip 414 is a two-stage soft tip plunger as described in Published PCT Patent Application No. WO2006059183, titled TWO STAGE PLUNGER FOR INTRAOCULAR LENS INJECTOR, by inventor J. Pynson, filed on Nov. 30, 2004. Plunger tip 414 has a relatively hard forked shaped tip 117 and a soft (compressible) sleeve 419. It is to be appreciated that in embodiments, in which the plunger tip is such a soft tip, it may be advantageous if the proximal end of the lumen portion 107b has a funnel shape (discussed below) to facilitate entry of the soft tip into the cartridge lumen 107, and to begin compression of the sleeve 419. Although injector 400 is illustrated as having a two-stage soft tip plunger, injectors for use with aspects of the present invention may have a conventional silicone soft tip or a fork-shaped hard tip plunger.

FIG. 6 is a perspective view of a partially assembled injector illustrating the cartridge 110 in the closed state and inserted into the proximal end 423 of the distal body portion 420 of the injector of FIG. 4A. FIG. 6 also illustrates that the cartridge lumen portion 107b has a funnel portion 101 on its proximal end to facilitate receiving of the plunger tip 414 into lumen 107 while the injector is being assembled.

The components of the injector shown in FIG. 4A may be assembled in any suitable order. Referring to FIG. 4A, according to one example of a suitable technique of assembly, cartridge 110 (in a closed state) is loaded into proximal end 423 of the distal body portion 420. Subsequently, proximal body portion 410 is attached to distal body portion 420, by sliding the distal body portion (with cartridge 110 disposed inside) into the proximal body portion 410. Proximal body portion 410 includes a plunger 412 having a thumb press 418. According to some embodiments, holder 175 (shown in FIG. 1) may be removably coupled to the proximal end 108 of the cartridge to facilitate loading of the cartridge into proximal end 423 of distal body portion 420 of the injector. After loading into proximal end 423, the handle is removed and the proximal body portion is connected to the distal body portion as described above.

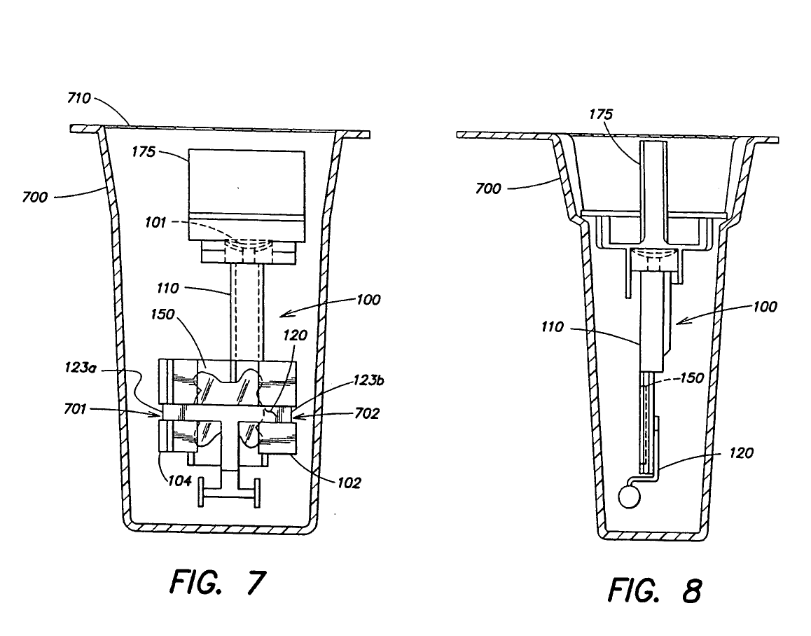

A subassembly 100 according to aspects of the present invention may be maintained in a sterile container prior to use. The container may be any suitable container. FIG. 7 and FIG. 8 are front and side views, respectively, of one example of an embodiment of a container 700 having the subassembly 100 of FIG. 1 disposed inside. In the illustrated embodiment, subassembly 100 includes notches 701 and 702 where the connectors 123a, 123b are attached. A foil cover 710 is provided to hold fluid inside the container and/or maintain sterility of the IOL. In the illustrated embodiment, holder 175 is connected to the cartridge to facilitate handling of the subassembly during injector assembly as described above.

Embodiments of subassemblies according to aspects of the invention may include, for example, a metal or plastic (e.g., polypropylene) cartridge. The fastener may be made of any suitable material, for example, polypropylene. The injector may be made of metal and/or plastic (e.g., polypropylene).

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An IOL injector subassembly, comprising:
(A) a lens cartridge comprising
(i.) a first portion comprising a first lumen segment, and
(ii.) a second portion comprising a second lumen segment, the second portion being rotatably connected to the first portion;
(B) an IOL; and
(C) a fastener connected to the lens cartridge arranged to maintain the IOL over the first lumen segment and over the second lumen segment.

2. The subassembly of claim 1, wherein the first portion and the second portion are configured to be capable of forming a first lumen, the sub-assembly further comprising a proximal portion fixedly coupled to the second portion and having a second lumen therethrough, the first lumen being aligned with a distal end of the second lumen.

3. The subassembly of claim 1, wherein the proximal end of the second lumen is funnel-shaped.

4. The subassembly of claim 1, wherein the fastener is connected to the cartridge at two locations.

5. The subassembly of claim 1, wherein the fastener is connected to the cartridge at four locations.

6. The subassembly of claim 1, wherein the cartridge comprises an injector tip configured for entry into an incision in an eye.

7. The subassembly of claim 1, in a combination with a container, wherein the sub-assembly is disposed in the container.

8. The combination of claim 7, wherein the container includes a cover to seal the subassembly in the container.

9. An IOL injector cartridge, comprising:
a first portion comprising a first lumen segment, and a second portion comprising a second lumen segment, the second portion being rotatably connected to the first portion, the first portion and the second portion forming a first lumen therebetween upon rotation of the first portion relative to the second portion;
a proximal portion fixedly coupled to the second portion and having a second lumen therethrough, the first lumen being aligned with the second lumen.

10. The cartridge of claim 9, wherein the proximal end of the second lumen is funnel-shaped.

11. The cartridge of claim 9, wherein the cartridge comprises an injector tip configured for entry into an incision in the eye.

12. A method for facilitating loading of an IOL injector, comprising:
(A) providing an IOL injector subassembly, the subassembly comprising
(I) a lens cartridge comprising,
(i.) a first portion comprising a first lumen segment, and
(ii.) a second portion comprising a second lumen segment, the second portion being rotatably connected to the first portion,
(II) an IOL, and
(III) a fastener connected to the lens cartridge arranged to maintain the IOL in a position over the first lumen segment and over the second lumen segment;
(B) removing the fastener; and
(c) rotating the first portion relative to the second portion to fold the IOL.

13. The method of claim 12, further comprising loading the cartridge into a distal body portion of the injector.

14. The method of claim 13, further comprising attaching the distal body portion to a proximal body portion of the injector.

15. The method of claim 13, wherein the step of loading the cartridge into a distal body portion of the injector comprises loading the cartridge into the distal body portion using a handle removably attached to the cartridge.

16. The method of claim 15, further comprising steps of removing the handle and attaching the distal body portion to a proximal body portion of the injector.
